(19) United States
(12) Patent Application Publication
(10) Pub. No.: US 2021/0180220 A1
(43) Pub. Date: Jun. 17, 2021

(54) DIET FUNCTIONAL FABRIC FOR BREAKING DOWN BODY FAT AND REDUCING WEIGHT

(71) Applicant: CELLOGIN INC., Wonju-si (KR)

(72) Inventors: Yeon Sook WEON, Goyang-si (KR); Han Sung KIM, Wonju-si (KR); Seung Kwan CHO, Goyang-si (KR); Seong Guk KIM, Wonju-si (KR); Chi Hwan LEE, Seongnam-si (KR); Ki Pyoung YANG, Daegu (KR)

(21) Appl. No.: 16/762,644

(22) PCT Filed: Nov. 23, 2017
(86) PCT No.: PCT/KR2017/013390

§ 371 Date: May 8, 2020

(30) Foreign Application Priority Data
Nov. 14, 2017 (KR) 10-2017-0151716

(51) Int. Cl. D03D 1/00; D06M 11/74; D06M 15/564; D06M 11/05

(52) U.S. Cl. CPC D03D 1/0088; D06M 11/74; D10B 2501/00; D06M 11/05; D06M 15/564

(57) ABSTRACT

A functional fabric in which a microcurrent is applied according to the present invention comprises water-dispersion polyurethane, graphite, water (H₂O), and a thickener.

### TECHNICAL FIELD

The present invention relates to a diet functional fabric for body fat and weight loss.

### BACKGROUND ART

Micro biological currents flowing the human body deliver information between the cerebrum and internal organs to allow it to stay healthy.

Such micro biological currents become weak and unstable when one is unhealthy. Recent research shows that artificial application of micro currents to the human body provides beneficial effects.

For example, micro current stimulation to the human body provides various effects, such as fatigue recovery, pain relief, skin enhancement, anti-aging, physis stimulation, germ suppression, wound treatment, vision recovery, or weight care and thus has various applications.

Meanwhile, new material functional clothing is being developed which helps boosting metabolism and, as customers demand for healthy and sanitary life increases, healthy, functional fibers are being introduced.

Examples of such functional fibers include natural mineral fibers, charcoal fibers, vitamin fibers, silver fibers, fibers capable of radiating far infrared light, and other various types of skin care fibers containing vitamin C, green tea extraction, or other effective substances.

Despite various research and development efforts, however, no or little is known about the effects on the human metabolism of clothing made of such functional substances, and a further study is needed on brand-new substances being steadily developed.

In particular, although functional substances which work well for losing body fat and weight are developed and applied to fibers, such effective substances may be washed out by repeated washing, losing their functionality. Furthermore, there is no continuous discussion underway to ensure durability and better economy in the manufacturing process.

Korean Patent No. 10-963326 discloses a method for manufacturing a functional fabric for losing fat and weight, which may gain diet effects and durability and functional clothing manufactured by the method.

However, the prior art fails to evenly radiate wave energy through the functional fabric, so that the wearer of the functional fabric-applied clothes may not precisely set up and transfer micro currents for obtaining her desired effects, such as muscle growth and fat loss.

### PRIOR TECHNICAL DOCUMENTS

### Patent Documents

(Patent Document 0001) KR 10-0963326 B

### DETAILED DESCRIPTION OF INVENTION

### Technical Problems

To provide the user's desired effects, an embodiment of the present invention is to provide a diet functional fabric for breaking down body fat and losing weight, which allows micro currents, set up and supplied from the outside to provide effects, to be precisely delivered to the skin, muscles, and other body tissue.

However, the objects of the embodiments are not limited thereto, and other objects may also be present.

### Means to Address the Problems

As a technical means to achieve the foregoing technical objects, according to a first aspect of the present invention, a functional fabric to which micro current is applied comprises 10 parts by weight to 50 parts by weight of graphite, 10 parts by weight to 30 parts by weight of water (H2O), and one part by weight to three parts by weight of a thickener, relative to 100 parts by weight of water-dispersion polyurethane.

As the water-dispersion polyurethane, graphite, water, and thickener are stirred at 2,500 rpm to 4,000 rpm, a viscosity 2,000 cps to 5,000 cps is formed.

The micro current ranges from 10 μA to 1,000 μA.

The functional fabric is implemented as one of a form bondable to a fiber fabric, a form coatable or impregnatable to the fiber fabric, and a form applied to the fiber fabric and is coupled with the fiber fabric.

As the micro current is applied to a position where the user wears the functional fabric, the functionalfabric obtains an effect of body care, a diet for body fat loss and weight loss, skin care, skin disease enhancement, pain relief, musculoskeletal disorder enhancement or musculoskeletal system growth.

### Effects of Invention

By any one of the means to achieve the goals as described above, micro currents supplied from the outside may be transferred to the body tissue in a precise and stable manner, maximizing the user's desired effects.

Further, upon wearing the functional fabric-applied clothes, the user may lose body fat and thus have a diet effect.

### BRIEF DESCRIPTION OF DRAWINGS

FIG. 1A is a graph illustrating the results of the experiment regarding the abdominal fat volume of each group.

FIG. 1B is a graph comparing the respective abdominal fat volume variations of the groups.

FIG. 2 is a graph illustrating the results of an experiment for enhancing skin wrinkles with the worn product of the present invention.

FIG. 3 illustrates example photos showing the degree of enhancement in the skin disease by a worn product of the present invention.

FIG. 4 is a graph illustrating the degree of low back pain relief by a worn product of the present invention.

FIG. 5 is a graph illustrating variations in height by a worn product of the present invention.

### BEST MODE TO PRACTICE INVENTION

Hereinafter, embodiments of the present invention are described in detail to be easily practiced by one of ordinary skill in the art to which the present invention pertains with reference to the accompanying drawings. However, the present invention may be implemented in other various forms and is not limited to the embodiments set forth herein. For clarity of the disclosure, irrelevant parts are removed from the drawings, and similar reference denotations are used to refer to similar elements throughout the specification.

Throughout the specification, when an element is “connected” with another element, the element may be “directly connected” with the other element, or the element may be “electrically connected” with the other element via an intervening element.

Throughout the specification, when one member is positioned “on” another member, the first member may be positioned directly on the second member, or other member(s) may be positioned between the first and second member.

When an element “includes” another element, the element may further include the other element, rather excluding the other element, unless particularly stated otherwise. When the measurement of an element is modified by the term “about” or “substantially,” if a production or material tolerance is provided for the element, the term “about” or “substantially” is used to indicate that the element has the same or a close value to the measurement and is used for a better understanding of the present invention or for preventing any unscrupulous infringement of the disclosure where the exact or absolute numbers are mentioned. As used herein, “step of” A or “step A-ing” does not necessarily mean that the step is one for A.

The present invention relates to a diet functional fabric for decomposing fat and losing weight and a functional fabric to which micro currents are applied.

According to the present invention, a functional fabric to which micro currents are applied includes water-dispersion polyurethane, graphite, water (H₂O), and a thickener.

The functional fabric to which micro currents are applied includes 10 parts by weight to 50 parts by weight of the graphite, 10 parts by weight to 30 parts by weight of the water, and 1 part by weight to 3 parts by weight of the thickener, relative to 100 parts by weight of the water-dispersion polyurethane.

At this time, as the thickener, e.g., hydrophobically modified ethylene oxide urethane (HEUR) may be used.

When the content of the graphite is less than 10 parts by weight, the electrical conductance is low and, when the content of the graphite is more than 50 parts by weight, the electrical conductance is high but, upon washing, the graphite may be peeled off or removed, deteriorating the durability of the fabric. The water and the thickener have a relationship of being proportional with each other. When the content of the water is less than 10 parts by weight, the viscosity increases but becomes uneven and, when the content of the water is more than 30 parts by weight, the viscosity decreases and more infiltration occurs, thinning the film. When the content of the thickener is less than 1 part by weight, the viscosity decreases, more infiltration occurs, and the thickness of the film decreases. When the content of the thickener is more than 3 parts by weight, the viscosity increases but becomes uneven.

As the water-dispersion polyurethane, graphite, water, and thickener are stirred at 2,500 rpm to 4,000 rpm, the viscosity becomes 2,000 cps to 5,000 cps. At this time, if the viscosity of the mixture of the water-dispersion polyurethane, graphite, water, and thickener is 2,000 cps or less, it is difficult to adjust the thickness upon manufacturing the functional fabric and, if the viscosity is 5,000 cps or more, adjustment of thickness id easy but, upon drying, pin holes may be created.

The functional fabric formed with the mixture of the water-dispersion polyurethane, graphite, water, and thickener may receive micro currents ranging from 100 to 1,0000.

At this time, according to an embodiment of the present invention, the functional fabric is formed of a mixture of the water-dispersion polyurethane, graphite, water, and thickener and may thus receive micro currents supplied from the outside, as they are, and transfer the micro currents to the body tissue.

For example, in the functional fabric according to an embodiment of the present invention, the magnitude of micro current optimized for diet effects is 50 μA and, if 50 μA of micro current is supplied from an external power supply, the functional fabric may stably transfer 50 μA of current to the body tissue, providing for a desired diet effect.

As another example, in the functional fabric according to an embodiment of the present invention, the magnitude of micro current optimized for enhancing muscle elasticity is 4000 μA and, if 4000 μA of micro current is supplied from an external power supply, the functional fabric may stably transfer 4000 μA of current to the body tissue, providing for a desired effect for enhancing muscle elasticity.

Meanwhile, the micro current-applied functional fabric, according to an embodiment of the present invention, may be formed in the form of a film to be bonded to a fiber fabric. Or, the functional fabric may be formed in an applicable form and be applied onto a fiber fabric, or the functional fabric may be formed in a coatable or impregnatable form, and a fiber fabric may be coated or impregnated with the functional fabric. The film form, applicable form, and coatable form may be properly combined and applied depending on the function or kind of the fiber fabric or clothing to which the functional fabric is applied.

In an example in which a functional fabric is coated with the functional fabric, the mixture is directly applied, in a thickness of 20 μm to 500 μm, onto a release sheet for making films, using, e.g., floating-type or roll on-type coating machine and is then dried at 70° C. to 100° C. in an air dryer for two minutes to five minutes.

At this time, as the release sheet, a typical release sheet may be used, and the thickness of the release sheet may be adjusted to increase the period of reuse of the release sheet. When the thickness of the mixture applied onto the release sheet is less than 20 μm, the strength of the fabric is weak and may be damaged and, when the thickness of the mixture is more than 500 μm, the manufacturing cost rises and the flexibility of the fabric may decrease.

When the drying temperature is more than 100° C., the productivity may increase but pin holes may be created, making the conductivity uneven and hence resulting in quality deterioration.

The film formed on the release sheet is attached to a fiber fabric and then the release sheet is removed.

The product of the film and the fiber fabric attached together is cured at 110° C. to 130° C. for two minutes to five minutes, using a tenter to stay stable in shape.

Meanwhile, various kinds of fiber fabrics may be used. Specifically, natural fibers, such as linen, silk, or cotton, synthetic fibers, such as nylon, polyester, polyacrylic, potyamide, or polyvinyl chloride, regenerated fibers, such as rayon or acetate, and mixtures thereof may be used.

Meanwhile, according to an embodiment of the present invention, the functional fabric to which micro currents are applied may be provided in the form of clothes which are tightly worn on the skin.

The power supply which applies micro currents may be electrically connected with the clothes via a connector which is connected with the clothes to be able to apply the micro currents to the wearer's muscles and skin.

The power supply may have a display panel on the front surface thereof to turn on or off application of the micro currents or adjust the strength of the micro currents and a coupling hook on the back surface thereof to couple or decouple to/from the clothes in the user's desired position. Various sizes and shapes may be selected for the power supply in the process of manufacture.

Meanwhile, the clothes may include a body portion and an insulator portion. The body portion may be formed in the form of coming in surface contact, and sticking, to the skin. The insulator portion is provided to partition the body portion into an even number of segments.

Further, the body portion has a coupler that connects to the connector of the power supply. Thus, the connector includes a pair of a first connector, through which current of one polarity selected between + and − is applied, and a second connector, through which current of the opposite polarity of the first connector is applied and is configured such that one connector is connected with one coupler.

The coupler is formed to be broken in a certain position of the body portion, and the connector is formed to be inserted to the coupler while contacting the body portion, Meanwhile, the connector may be formed in various shapes, e.g., a clamp, pin, or clip, to be able to connect to the coupler.

According to an embodiment of the present invention, superior flexibility and elasticity, smooth texture, and high washing durability may be expected.

Further, as micro currents are applied to the position where the user wears the functional fabric, one or more effects among body care, diet, skin care, skin trouble enhancement, pain relief, musculoskeletal disorder enhancement, and musculoskeletal system growth may be expected.

Experimental examples for the above-described effects are described below in detail.

Experiment 1. Body Care and Diet

Of female testees in their thirties to fifties, 20 with similar weight and metabolism were selected and were randomly divided into an obese group (HFD, n=5) and micro current-applied obese groups (HFD+22 μA, n=5; HFD+50 μA, n=5; HFD+100 μA, n=5).

All of the groups were asked to go on a high fat diet for six weeks after an adaptation period (one week) to cause obesity.

The micro current stimulation used for this experiment were 22 μA, 50 μA, and 100 μA, and the experiment was performed one hour per day and five days per week for four weeks to fit the respective parameters of the groups.

The results of the experiment were indicated with mean±standard deviation (SD), and Prism 5.0, (Graphpad software, USA), as statistical analysis, was used, and one-way ANOVA was performed to compare statistical significances for the effects of micro current stimulation.

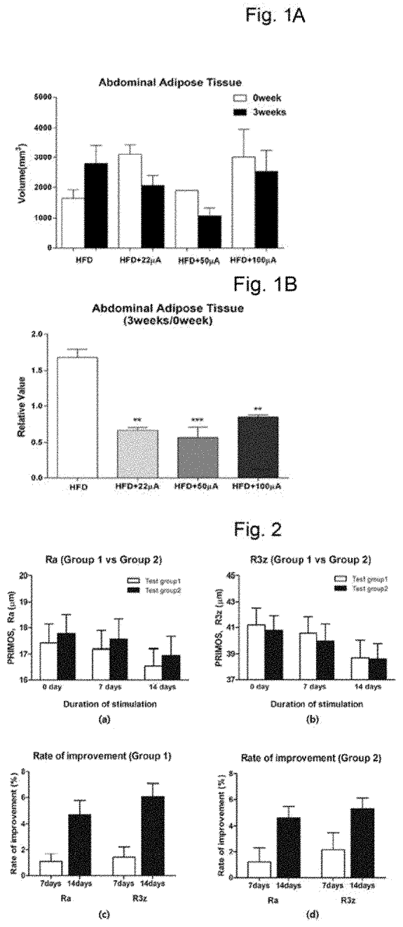

FIG. 1A is a graph illustrating the results of the experiment regarding the abdominal fat volume of each group. FIG. B is a graph comparing the respective abdominal fat volume variations of the groups.

Referring to FIG. 1A, it may be identified from the results of the experiment that the HFD group exhibited an increase in the abdominal adipose tissue volume but the other three groups exhibited a decrease in the abdominal adipose tissue.

It was also identified that, three weeks later, it was increased 1.68 times more as compared with the zeroth week, as shown in FIG. 1B, revealing that fat accumulation continued to increase due to the high fat diet. In contrast, the abdominal adipose tissue volume was reduced 0.66 times for HFD+22 μA, 0.57 times for HFD+50 μA, and 0.85 times for HFD+100 μA and, from these results, it was identified that micro current stimulation works well for losing fat.

It was also identified that fat loss was achieved for all of the three stimulated groups and, among them, the HFD+50 μA group exhibited a relatively significant reduction as compared with the increase for the HFD.

Experiment 2. Skin Care (Anti-Wrinkle)

20 female adults at age of 35 to 55, who were healthy and free of an acute or chronic disease but has a skin disease, were chosen as testees.

Before the experiment, with the test region cleansed and then dried up, the testees were allowed to relax for skin in a room that remains at a constant temperature (22±2° C.) and constant humidity (relative humidity of 40% to 60%) for, at least, 30 minutes. At that time, the test region was limited to around the eyes, and the clothing portion, which was made in the form of a band to tightly contact the surroundings of the eyes, was used for the experiment.

The lines and wrinkles around the eyes were measured on the region around the eyes (test region) using PRIMOS(Phaseshift Rapid In-vivo Measurement Of skin, GFM, GerAny) and, to verify the degree of wrinkle enhancement on the same test region, an overlay method was used. to measure on the same test region.

As PRIMOS analysis variables, the arithmetic roughness average (Ra) and the base roughness depth (R3z) were chosen. Ra is the arithmetic average of the maximum and minimum values for the roughness profile of the measured wrinkles, and Ra being smaller means that the depth of the skin wrinkles reduce and are thus mitigated. R3z is the arithmetic average for the five single roughness depths of R3z1 to R3z5, and R3z being smaller means that the base depth of the skin wrinkles reduces and the skin wrinkles are thus mitigated.

Meanwhile, the wrinkles-around-the-eyes enhancement rate (%) was calculated by (− variation (μm) in wrinkles around the eyes before and after use of the product)/(measurement (μm) of the wrinkles around the eyes before use of the product)×100.

FIG. 2 is a graph illustrating the results of an experiment for enhancing skin wrinkles with the worn product of the present invention. It is identified from FIG. 2 that the depth of the wrinkles around the testee's eyes decreases and thus the present invention works for wrinkle enhancement.

Experiment 3. Skin Disease Enhancement

One male adult with erythema on his back was selected as a testee. The clothing portion was made to be able to be overall tightly worn on the testee's back, and was worn on the testee, and in that state, micro currents were applied one hour per day for three months, and then the erythema was observed with the naked eye.

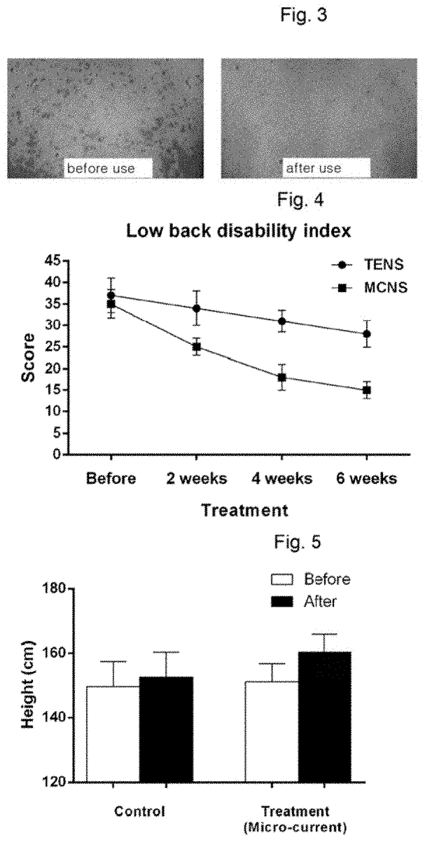

FIG. 3 illustrates example photos showing the degree of enhancement in the skin disease by a worn product of the present invention. Referring to FIG. 3, it may be identified that the testee's region of skin disease was enhanced.

Experiment 4. Musculoskeletal Disorder Enhancement (Lower Back Pain Relief)

As testees, 10 females at age of 50 to 65, with chronic low back pain were chosen, and the clothing portion was made to wrap around the waist.

Treatment group 1: For the testees, micro currents were applied five hours per day for six weeks and the Oswestry disability index was assessed (MCNS: Micro current nerve stimulation)

Treatment group 2: Three days after treatment group 1 was performed, for the same testees, transcutaneous electrical nerve stimulation was applied five hours per day for six weeks and the Oswestry disability index was assessed. (TENS : Transcutaneous electrical nerve stimulation)

FIG. 4 is a graph illustrating the degree of low back pain relief by a worn product of the present invention. It is identified that treatment group 1, to which micro currents are applied, exhibits a better pain relief effect than treatment group 2, to which transcutaneous electrical nerve stimulation is applied.

Experiment 5. Museuloskeletal System Growth (Variation in Height)

10 children at age of 12 to 13 were selected as testees. The clothing portion was made to wrap around the testees' knee.

Treatment group 1 (treatment): Five were selected from among the testees and were let wear the clothing portion, and micro currents were applied to the five testees, three hours per day for three months, and variations in height were measured.

Treatment group 2 (control): The other five testees than those in treatment group 1 were measured for variations in height after three months elapsed.

At that time, the experiment was performed in the same place under the same accommodation conditions for treatment group 1 and treatment group 2 so as to determine influences only from micro currents, and the amount of movement was limited similarly.

FIG. 5 is a graph illustrating variations in height by a worn product of the present invention. It may be identified that treatment group I exhibits a relatively high growth rate as compared with treatment group 2.

Although embodiments of the present invention have been described with reference to the accompanying drawings, It will be appreciated by one of ordinary skill in the art that the present disclosure may be implemented in other various specific forms without changing the essence or technical spirit of the present disclosure. Thus, it should be noted that the above-described embodiments are provided as examples and should not be interpreted as limiting. Each of the components may be separated into two or more units or modules to perform its function(s) or operation(s), and two or more of the components may be integrated into a single unit or module to perform their functions or operations.

It should be noted that the scope of the present invention is defined by the appended claims rather than the described description of the embodiments and include all modifications or changes made to the claims or equivalents of the claims.

1. A functional fabric to which micro current is applied, the functional fabric comprising 10 parts by weight to 50 parts by weight of graphite, 10 parts by weight to 30 parts by weight of water (H2O), and one part by weight to three parts by weight of a thickener, relative to 100 parts by weight of water-dispersion polyurethane.

2. The functional fabric of claim 1, wherein as the water-dispersion polyurethane, graphite, water, and thickener are stirred at 2,500 rpm to 4,000 rpm, a viscosity of 2,000 cps to 5,000 cps is formed.

3. The functional fabric of claim 1, wherein the micro current ranges from 10 μA to 1,000 μA.

4. The functional fabric of claim 1, wherein the functional fabric is implemented as one of a form bondable to a fiber fabric, a form coatable or impregnatable to the fiber fabric, and a form applied to the fiber fabric and is coupled with the fiber fabric.

5. The functional fabric of claim 1, wherein as the micro current is applied to a position where the user wears the functional fabric, an effect of body care, a diet for body fat loss and weight loss, skin care, skin disease enhancement, pain relief, musculoskeletal disorder enhancement or musculoskeletal system growth is obtained.
